(19) United States
(12) Patent Application Publication
(10) Pub. No.: US 2014/0114183 A1
(43) Pub. Date: Apr. 24, 2014

(54) COMPUTERIZED TOMOGRAPHY (CT) FLUOROSCOPY IMAGING SYSTEM USING A STANDARD INTENSITY CT SCAN WITH REDUCED INTENSITY CT SCAN OVERLAYS

(71) Applicant: NeuroLogica Corp., Danvers, MA (US)

(72) Inventor: Eric Bailey, North Hampton, NH (US)

(21) Appl. No.: 14/059,120

(22) Filed: Oct. 21, 2013

Related U.S. Application Data
(60) Provisional application No. 61/716,341, filed on Oct. 19, 2012.

(51) Int. Cl. A61B 6/03; A61B 6/00

(52) U.S. Cl. CPC A61B 6/032; A61B 6/481; A61B 6/485
USPC 600/431

(57) ABSTRACT

A method for providing CT fluoroscopy of an internal procedure site during a medical procedure so as to visualize patient anatomy and a high-contrast medical element during the procedure without subjecting the patient to unacceptable quantities of X-ray radiation, comprising: (1) taking a standard intensity CT scan of the internal procedure site; (2) taking a reduced intensity CT scan of the procedure site while the high-contrast medical element is inserted into the procedure site; (3) extracting the high-contrast medical element portion of the reduced intensity CT scan from the reduced intensity CT scan; (4) merging the high-contrast medical element portion of the reduced intensity CT scan extracted in Step 3 with the standard intensity CT scan of the procedure site taken in Step 1 so as to provide a composite CT scan; (5) displaying the composite CT scan; and (6) returning to either Step 1 or Step 2.

### REFERENCE TO PENDING PRIOR PATENT APPLICATION

This patent application claims benefit of pending prior U.S. Provisional Patent Application Ser. No. 61/716,341, filed Oct. 19, 2012 by Eric Bailey for COMPUTERIZED TOMOGRAPHY (CT) FLUOROSCOPY IMAGING SYSTEM USING HIGH DOSE SCAN WITH LOW DOSE OVERLAY (Attorney's Docket No. NEUROLOGICA-10 PROV), which patent application is hereby incorporated herein by reference.

### FIELD OF THE INVENTION

This invention relates to anatomical imaging systems in general, and more particularly to Computerized Tomography (CT) imaging systems.

### BACKGROUND OF THE INVENTION

Computerized Tomography (CT) has emerged as a key imaging modality in the visualization of anatomy. CT scanners generally operate by directing X-rays into the body from a variety of positions, detecting the X-rays passing through the body, and then processing the detected X-rays so as to build a three-dimensional (3D) computer model of the patient's anatomy. This 3D computer model can then be visualized (e.g., as a 3D visualization or as individual “slice” visualizations) so as to provide images of the patient's anatomy. See, for example, FIG. 1, which shows a typical CT image of a patient's anatomy, wherein the anatomy is displayed as an individual “slice” visualization.

Ideally, it would be desirable to continuously image the patient's anatomy during a medical procedure so as to provide substantially continuous imaging information to the physician during the procedure. Such substantially continuous imaging could comprise a series of images taken in rapid succession or this continuous imaging could comprise live video imaging. Such substantially continuous imaging would allow the physician to observe changes in the patient's anatomy during the procedure, as well as to observe the position of instruments, prostheses, etc. vis-à-vis the patient's anatomy.

Such an approach is frequently used with conventional two-dimensional (2D) X-ray machines, and is sometimes referred to as “fluoroscopy”.

Unfortunately, CT machines, since they must direct X-rays into the body from a variety of positions, emit significantly higher quantities of radiation during imaging than conventional 2D X-ray machines. Therefore, it is generally impractical to operate a conventional CT machine substantially continuously during a medical procedure, whereby to provide 3D fluoroscopy during the medical procedure, since the quantity of radiation which would be emitted during the medical procedure is generally deemed unacceptable.

Furthermore, operating a conventional CT machine at significantly reduced X-ray intensities so as to provide 3D fluoroscopy is generally not a viable option, since scanning anatomy with significantly reduced X-ray intensities generally results in an inferior image, particularly where low-contrast (e.g., soft tissue) anatomy is involved. See, for example, FIG. 2, which is a schematic view illustrating how imaging low-contrast (e.g., soft tissue) anatomy with significantly reduced X-ray intensities results in a degraded image.

Thus, there is a need for a new and improved CT system which would provide 3D fluoroscopy of an internal procedure site during a medical procedure so as to visualize patient anatomy, medical instruments, prosthesis, etc. during the medical procedure without subjecting the patient to unacceptable quantities of X-ray radiation.

### SUMMARY OF THE INVENTION

In accordance with the present invention, there is provided a novel CT system capable of providing 3D fluoroscopy of an internal procedure site during a medical procedure so as to visualize patient anatomy, medical instruments, prosthesis, etc. during the medical procedure without subjecting the patient to unacceptable quantities of X-ray radiation. This new CT system provides 3D fluoroscopy of the internal procedure site using a standard intensity CT scan with reduced intensity CT scan overlays.

In one preferred form of the invention, there is provided a method for providing CT fluoroscopy of an internal procedure site during a medical procedure so as to visualize patient anatomy and a high-contrast medical element during the medical procedure without subjecting the patient to unacceptable quantities of X-ray radiation, the method comprising:

(1) taking a standard intensity CT scan of the internal procedure site;

(2) taking a reduced intensity CT scan of the internal procedure site while the high-contrast medical element is inserted into the internal procedure site;

(3) extracting the high-contrast medical element portion of the reduced intensity CT scan from the reduced intensity CT scan taken in Step 2;

(4) merging the high-contrast medical element portion of the reduced intensity CT scan extracted in Step 3 with the standard intensity CT scan of the internal procedure site taken in Step 1 so as to provide a composite CT scan;

(5) displaying the composite CT scan generated in Step 4; and

(6) returning to either Step 1 or Step 2.

In another preferred form of the invention, there is provided apparatus for providing CT fluoroscopy of an internal procedure site during a medical procedure so as to visualize patient anatomy and a high-contrast medical element during the medical procedure without subjecting the patient to unacceptable quantities of X-ray radiation, the apparatus comprising:
a CT machine configured to:
   (1) take a standard intensity CT scan of the internal procedure site;
   (2) take a reduced intensity CT scan of the internal procedure site while the high-contrast medical element is inserted into the internal procedure site;
   (3) extract the high-contrast medical element portion of the reduced intensity CT scan from the reduced intensity CT scan taken in Step 2;
   (4) merge the high-contrast medical element portion of the reduced intensity CT scan extracted in Step 3 with the standard intensity CT scan of the internal procedure site taken in Step 1 so as to provide a composite CT scan;
   (5) display the composite CT scan generated in Step 4; and
   (6) return to either Step 1 or Step 2.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a typical CT scan of a patient's anatomy, with the image being taken using X-rays of standard CT scan intensity;

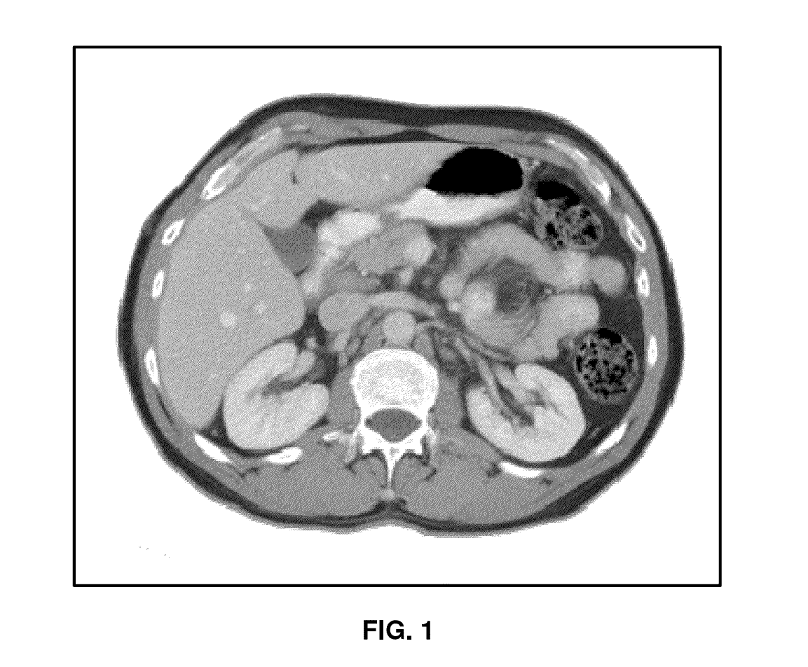

FIG. 2 is a schematic view showing a CT scan of a patient's anatomy where the image is taken using X-rays of reduced CT scan intensity;

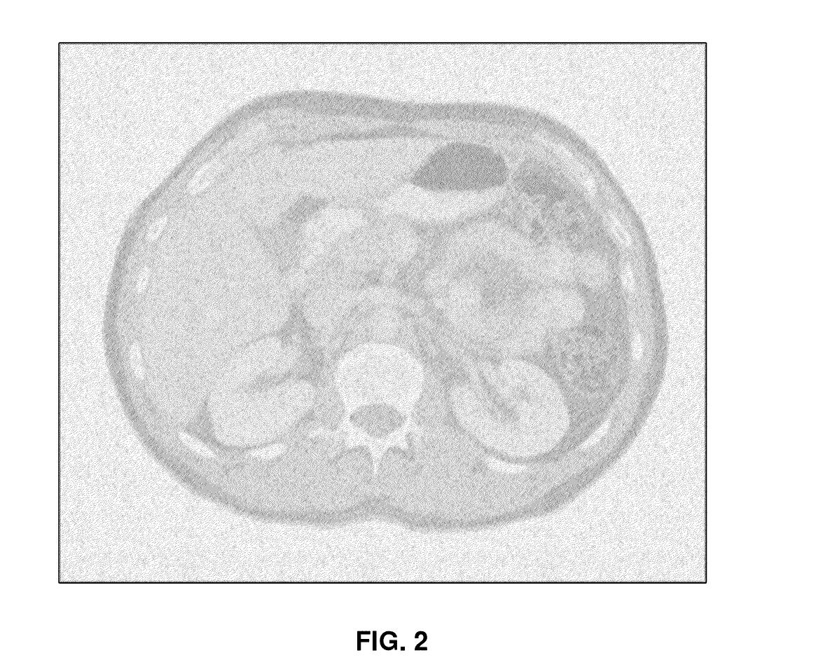

FIG. 3 is a schematic view showing a CT scan of a patient's anatomy and a high-contrast medical element inserted into the patient's anatomy, where the image is taken using X-rays of standard CT scan intensity;

FIG. 4 is a schematic view showing a CT scan of a patient's anatomy and a high-contrast medical element inserted into the patient's anatomy, where the CT scan is taken using X-rays of reduced CT scan intensity;

FIG. 5 is a schematic view showing a CT scan of a patient's anatomy where the CT scan is taken using X-rays of standard CT scan intensity;

FIG. 6 is a schematic view showing a CT scan of a patient's anatomy and a high-contrast medical element inserted into the patient's anatomy, where the CT scan is taken using X-rays of reduced CT scan intensity;

FIG. 7 is a schematic view showing the CT scan of the high-contrast medical element of FIG. 6 after it has been isolated from the remainder of the CT scan of FIG. 6;

FIG. 8 is a schematic view showing the extracted CT scan of the high-contrast medical element of FIG. 7 merged with the standard intensity CT scan of the anatomy (FIG. 5) so as to provide a composite CT scan; and

FIG. 9 is a flowchart of the novel method of the present invention for providing 3D fluoroscopy of an internal procedure site during a medical procedure.

### DETAILED DESCRIPTION OF THE PREFERRED EMBODIMENTS

The present invention provides a novel CT system capable of providing 3D fluoroscopy of an internal procedure site during a medical procedure so as to visualize patient anatomy, medical instruments, prostheses, etc. during the medical procedure without subjecting the patient to unacceptable quantities of X-ray radiation. This new CT system provides 3D fluoroscopy of the internal procedure site using a standard intensity CT scan with reduced intensity CT scan overlays.

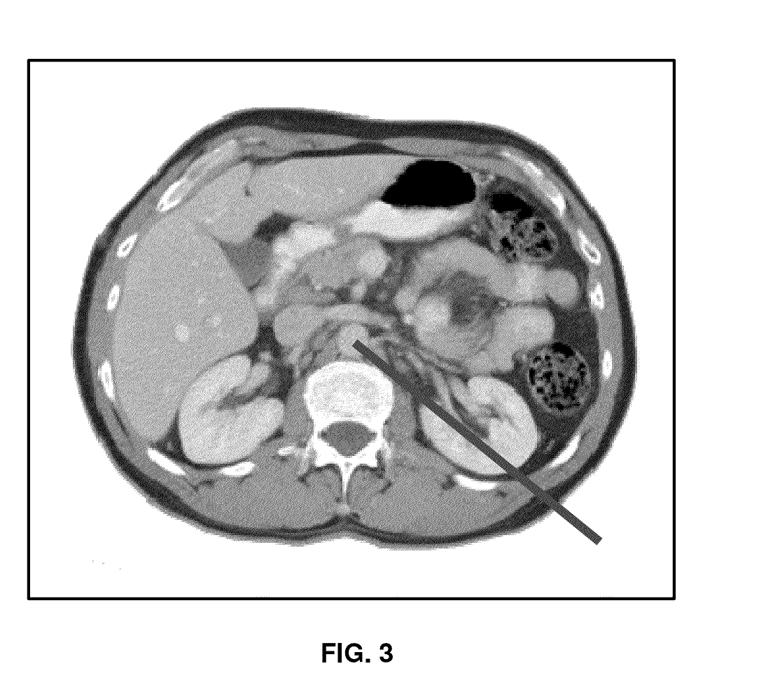

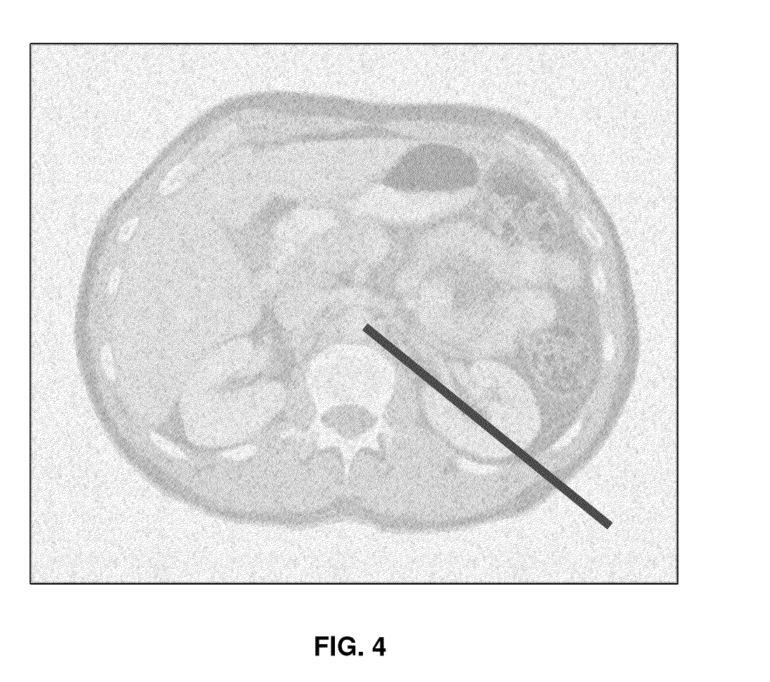

More particularly, it has been noted that certain medical elements (e.g., surgical instruments, prostheses, catheters, needles, injectable substances such as iodine, etc.) are relatively high-contrast elements which are capable of being accurately visualized using lower X-ray intensities than is generally necessary in order to accurately visualize low-contrast (e.g., soft tissue) anatomy. Thus, for example, FIG. 3 shows a high-contrast surgical instrument disposed within the anatomy imaged by a CT machine operating at standard CT X-ray intensities (which, for the purposes of the present invention, may be considered to be standard intensity CT scan intensities), and FIG. 4 shows the same high-contrast surgical instrument disposed within the anatomy imaged by a CT machine operating at a reduced X-ray intensity, i.e., an X-ray intensity sufficient to effectively image the high-contrast surgical instrument but too low to effectively image the anatomy itself (and which, for the purposes of the present invention, may be considered to be reduced intensity CT scan intensities). Note that visualization of the high-contrast surgical instrument is substantially undiminished in the image produced by the CT machine operating at reduced CT scan intensites (i.e., FIG. 4).

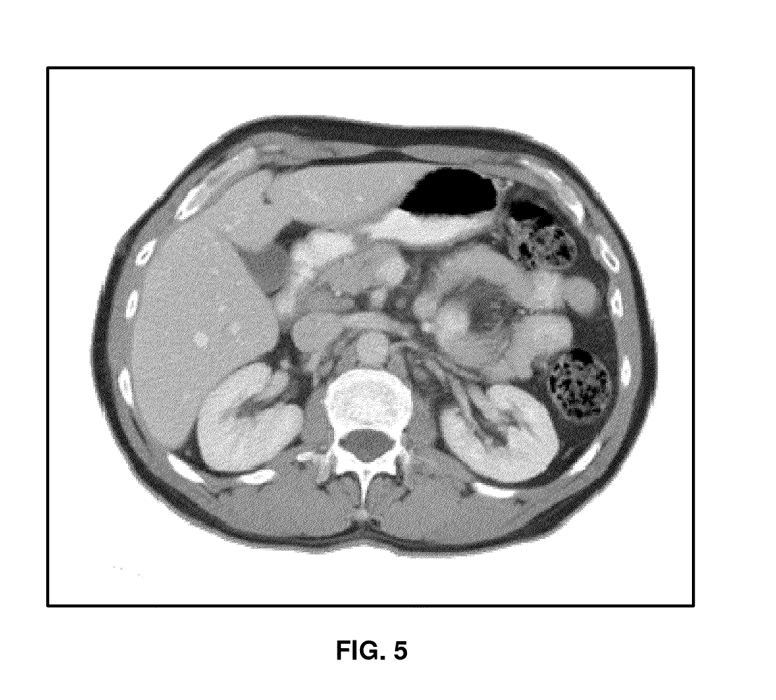

In accordance with the present invention, the CT fluoroscopy system of the present invention first takes a standard intensity CT scan of the internal procedure site before the high-contrast medical element (e.g., surgical instrument, prosthesis, catheter, needle, injectable substances such as iodine, etc.) is inserted into the surgical field. See FIG. 5. By way of example but not limitation, this standard intensity CT scan may be taken at an X-ray intensity level of approximately 300 mAs (milliamp seconds) for an adult brain.

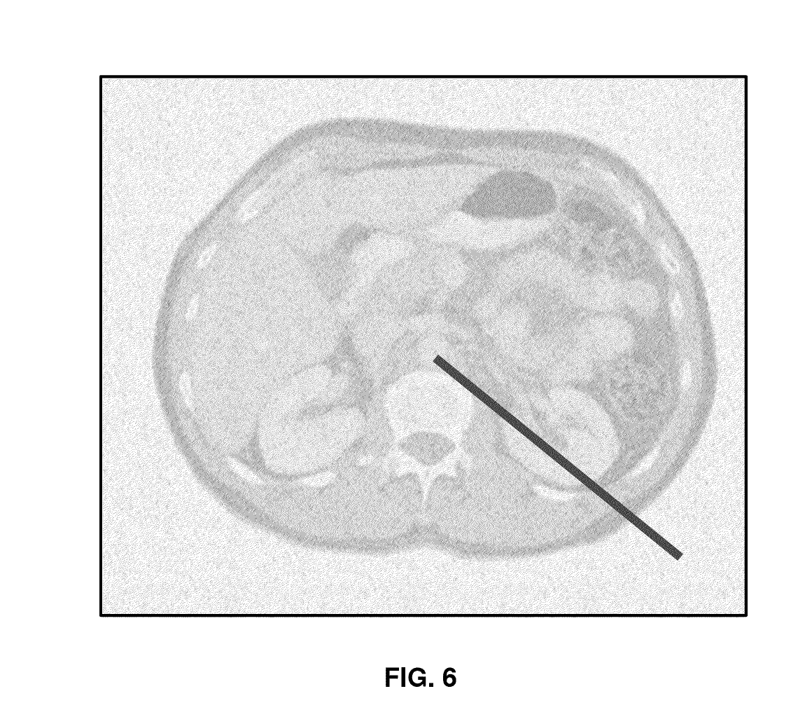

Thereafter, the CT fluoroscopy system takes a reduced intensity CT scan of the internal procedure site while the high-contrast medical element is inserted into the internal procedure site. See FIG. 6. As noted above, this image provides accurate visualization of the high-contrast medical element while providing poor visualization of low-contrast (e.g., soft tissue) anatomy. By way of example but not limitation, this reduced intensity CT scan may be taken at an X-ray intensity level of approximately 15-30 mAs (e.g., an X-ray intensity level which is only about 1/20 to 1/10 of the X-ray intensity of a standard intensity CT scan).

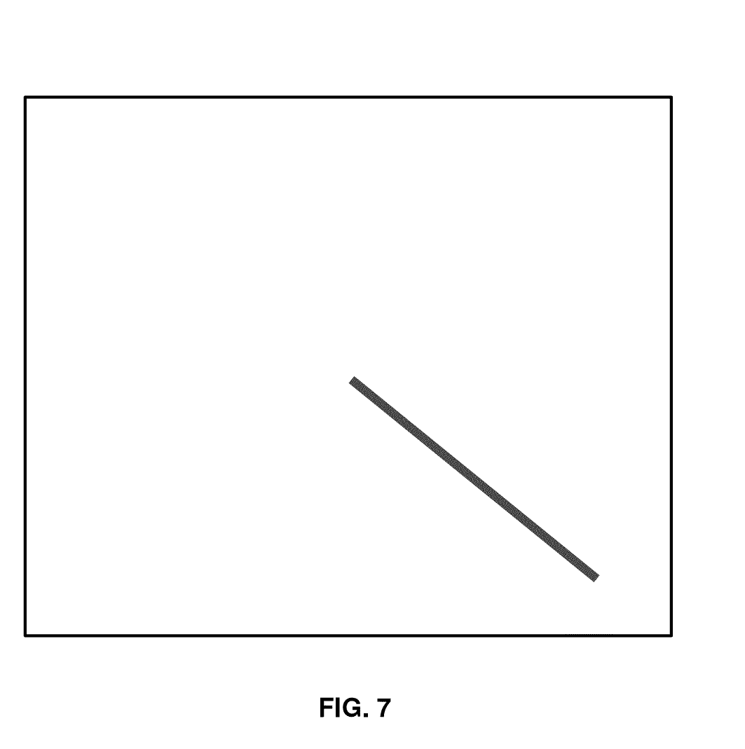

Next, the CT fluoroscopy system extracts the high-contrast medical element portion of the reduced intensity CT scan of FIG. 6. See FIG. 7, which shows the high-contrast medical element portion of the reduced intensity CT scan isolated from the remainder of the reduced intensity CT scan of FIG. 6. This process of isolation may be achieved by various ways well known in the art, e.g., by an image subtraction process, a segmentation process, etc.

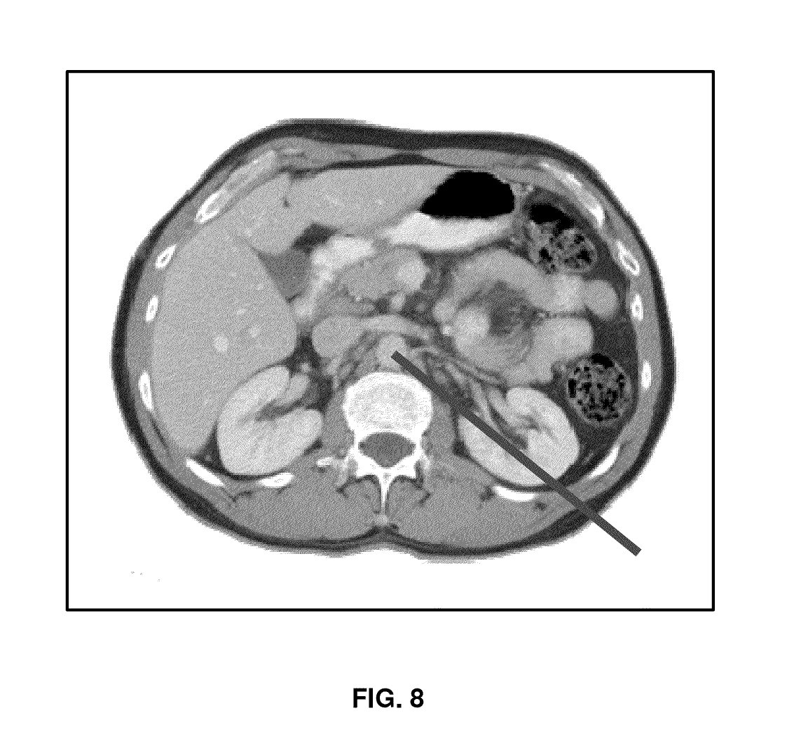

Then the CT fluoroscopy system merges (with appropriate element registration) (i) the extracted high-contrast medical element portion of the reduced intensity CT scan (FIG. 7) with (ii) the standard intensity CT scan of the internal procedure site (FIG. 5) so as to provide a composite CT scan (FIG. 8) which appropriately combines the accurate visualization of the high-contrast surgical instrument (achieved with a reduced intensity CT scan) with the accurate visualization of the anatomy (achieved with a standard intensity CT scan). This composite CT scan may then be displayed (e.g., to the physician conducting the procedure), recorded, etc.

Significantly, inasmuch as the high-contrast medical element is imaged with a reduced intensity CT scan, the high-contrast medical element may be scanned on a substantially continuous basis (e.g., as a series of scans taken in rapid succession or as a live video scan), even though the anatomy is imaged on a less regular basis, whereby to effectively provide 3D fluoroscopy of an internal procedure site during a medical procedure so as to visualize patient anatomy, medical instruments, prosthesis, etc. during the medical procedure without subjecting the patient to unacceptable quantities of X-ray radiation. Thus it is anticipated that multiple reduced intensity CT scans will be made of the internal procedure site for each standard intensity CT scan of the internal procedure site when a high-contrast medical element is moving about the internal procedure site. By way of example but not limitation, the high-contrast medical element may be scanned with a reduced intensity CT scan ten (10) times as often as the internal procedure site is scanned with a standard intensity CT scan. Thus it will be appreciated that the CT scan of the medical element is effectively “refreshed” 10 times as often as the CT scan of the internal procedure site.

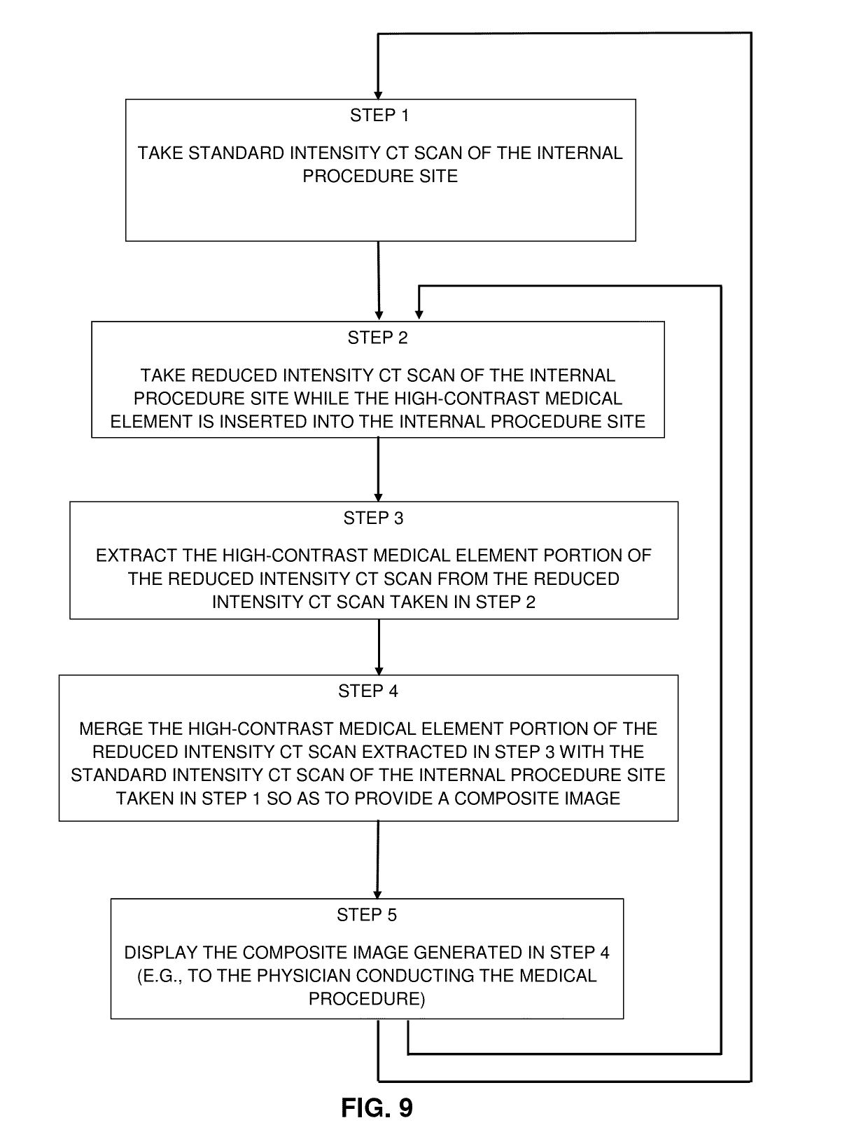

See FIG. 9, which provides a flow chart of the foregoing method for providing 3D fluoroscopy of an internal procedure site during a medical procedure.

### Modifications

It will be appreciated that still further embodiments of the present invention will be apparent to those skilled in the art in view of the present disclosure. It is to be understood that the present invention is by no means limited to the particular constructions herein disclosed and/or shown in the drawings, but also comprises any modifications or equivalents within the scope of the invention.

What is claimed is:

1. A method for providing CT fluoroscopy of an internal procedure site during a medical procedure so as to visualize patient anatomy and a high-contrast medical element during the medical procedure without subjecting the patient to unacceptable quantities of X-ray radiation, the method comprising:
(1) taking a standard intensity CT scan of the internal procedure site;
(2) taking a reduced intensity CT scan of the internal procedure site while the high-contrast medical element is inserted into the internal procedure site;
(3) extracting the high-contrast medical element portion of the reduced intensity CT scan from the reduced intensity CT scan taken in Step 2;
(4) merging the high-contrast medical element portion of the reduced intensity CT scan extracted in Step 3 with the standard intensity CT scan of the internal procedure site taken in Step 1 so as to provide a composite CT scan;
(5) displaying the composite CT scan generated in Step 4; and
(6) returning to either Step 1 or Step 2.

2. A method according to claim 1 wherein the standard intensity CT scan is taken at an X-ray intensity level of approximately 300 mAs.

3. A method according to claim 1 wherein the reduced intensity CT scan is taken at an X-ray intensity level of approximately 15-30 mAs.

4. A method according to claim 1 wherein the internal procedure site is scanned multiple times with a reduced intensity CT scan for each time the internal procedure site is scanned with a standard intensity CT scan.

5. A method according to claim 1 wherein the internal procedure site is scanned with a reduced intensity CT scan approximately 10 times as often as the internal procedure site is scanned with a standard intensity CT scan.

6. A method according to claim 1 wherein the high-contrast medical element comprises one from the group consisting of medical instruments, prostheses, catheters, needles and injectable substances.

7. Apparatus for providing CT fluoroscopy of an internal procedure site during a medical procedure so as to visualize patient anatomy and a high-contrast medical element during the medical procedure without subjecting the patient to unacceptable quantities of X-ray radiation, the apparatus comprising:
a CT machine configured to:
(1) take a standard intensity CT scan of the internal procedure site;
(2) take a reduced intensity CT scan of the internal procedure site while the high-contrast medical element is inserted into the internal procedure site;
(3) extract the high-contrast medical element portion of the reduced intensity CT scan from the reduced intensity CT scan taken in Step 2;
(4) merge the high-contrast medical element portion of the reduced intensity CT scan extracted in Step 3 with the standard intensity CT scan of the internal procedure site taken in Step 1 so as to provide a composite CT scan;
(5) display the composite CT scan generated in Step 4; and
(6) return to either Step 1 or Step 2.

8. Apparatus according to claim 7 wherein the standard intensity CT scan is taken at an X-ray intensity level of approximately 300 mAs.

9. Apparatus according to claim 7 wherein the reduced intensity CT scan is taken at an X-ray intensity level of approximately 15-30 mAs.

10. A method according to claim 7 wherein the internal procedure site is scanned multiple times with a reduced intensity CT scan for each time the internal procedure site is scanned with a standard intensity CT scan.

11. Apparatus according to claim 7 wherein the internal procedure site is scanned with a reduced intensity CT scan approximately 10 times as often as the internal procedure site is scanned with a standard intensity CT scan.

12. Apparatus according to claim 7 wherein the high-contrast medical element comprises one from the group consisting of medical instruments, prostheses, catheters, needles and injectable substances.
